(19) United States
(12) Patent Application Publication
(10) Pub. No.: US 2025/0295899 A1
(43) Pub. Date: Sep. 25, 2025

(54) SELF-TENSIONING IMPLANT

(71) Applicant: Teleflex Life Sciences LLC, Wilmington, DE (US)

(72) Inventors: Curtis Yarra, San Carlos, CA (US); Alexander Charles Gordon, Oakland, CA (US); Erik Noel, San Ramon, CA (US); Ernest Cabreza, Dublin, CA (US)

(73) Assignee: Teleflex Life Sciences LLC, Wilmington, DE (US)

(21) Appl. No.: 19/086,001

(22) Filed: Mar. 20, 2025

Related U.S. Application Data
(60) Provisional application No. 63/568,861, filed on Mar. 22, 2024.

(51) Int. Cl. A61M 29/00; A61B 17/34

(52) U.S. Cl. CPC A61M 29/00; A61B 17/3468; A61M 2205/0216; A61M 2210/166

(57) ABSTRACT

A prostatic implant configured to compress an enlarged prostate gland includes a coil member having a distal portion connected to a proximal portion by a middle portion. The distal portion and proximal portion each define a pre-formed coil structure configured to anchor the implant to a lateral lobe of the prostate gland while the middle portion extends therethrough in a partially uncoiled configuration. The implant, which may be a single member of unitary construction, is self-tensioning such that after implantation, the middle portion biases the distal portion and proximal portion toward each other, across the prostatic tissue. The enlarged anchoring footprints of the pre-formed coil structures prevent the distal and proximal portions from tearing through the prostatic tissue in response to the inherent tension force of the implant.

### CROSS-REFERENCE TO RELATED APPLICATIONS

This application claims the benefit of priority to U.S. Provisional Appln. Ser. No. 63/568,861, filed Mar. 22, 2024, which is incorporated herein by reference in its entirety.

### TECHNICAL FIELD

The subject matter of this patent document relates to the field of medical devices. More particularly, but not by way of limitation, the subject matter relates to medical devices, systems, and methods for relieving pressure on a prostatic urethra by compressing at least a portion of a prostate gland.

### BACKGROUND

Benign Prostatic Hyperplasia (BPH) is one of the most common medical conditions that affect men, especially elderly men. It has been reported that, in the United States, more than half of all men have histopathologic evidence of BPH by age 60 and, by age 85, approximately 9 out of 10 men suffer from the condition. Moreover, the incidence and prevalence of BPH are expected to increase as the average age of the population in developed countries increases. Despite extensive efforts in both the medical device and pharmacotherapeutic fields, current treatments remain only partially effective and are burdened with significant side effects. Thus, there remains a need for the development of new devices, systems, and methods for treating BPH as well as other conditions in which one tissue or anatomical structure impinges upon or compresses another tissue or anatomical structure.

### SUMMARY

Embodiments disclosed herein include devices, systems, and methods for compressing at least a portion of a prostate gland to alleviate pressure on a prostatic urethra. Embodiments include a coiled prostatic implant configured to anchor simultaneously to the outer prostatic capsule, and also a urethral side, of a lobe of an enlarged prostate, such as a median or lateral lobe. The implant may comprise a single member of unitary construction having distinct portions defined by different material properties and/or placement on or in a lobe upon completion of a treatment procedure described herein. For instance, a distal portion of the implant may assume a pre-formed coil structure after placement on the outer capsular surface of a lobe, a middle portion may assume a partially straightened, uncoiled structure extending through the lobe, and a proximal portion may assume a pre-formed coil structure after placement on a urethral side of the lobe. As a whole, the implant may be a self-tensioning device biased toward a coiled configuration such that, after implanting the device in a lobe, the distal and proximal portions anchored to opposing surfaces of the lobe are urged toward each other via the partially uncoiled middle portion in a manner that compresses the prostatic tissue therebetween. Relative to preexisting prostatic implants, the footprints of the distal and proximal anchors of the disclosed implant may be larger, thereby reducing the likelihood of the distal and proximal portions pulling or tearing through the prostatic tissue after implantation.

These and other examples and objects of the present devices, systems, and related methods will be set forth in the following Detailed Description. This Overview is intended to provide non-limiting examples of the present subject matter. The Detailed Description below is included to provide further information about the present devices and related methods. Neither is not intended to provide an exclusive or exhaustive explanation of the present devices and methods because this disclosure is written for those of ordinary skill in the art.

### BRIEF DESCRIPTION OF THE DRAWINGS

In the drawings, like numerals can be used to describe similar features and components throughout the several views. The drawings illustrate generally, by way of example but not by way of limitation, various embodiments discussed in the present patent document.

FIG. 1 illustrates a cross-sectional view of the anatomy surrounding a prostate in a human subject.

FIG. 2 illustrates an enlarged cross-sectional view of the anatomy surrounding a prostate.

FIG. 3 illustrates an implant in accordance with embodiments of the present disclosure.

FIG. 4 illustrates the implant shown in FIG. 3 after implantation in a prostate gland.

FIG. 5 illustrates a side view of a delivery device for an implant in accordance with embodiments of the present disclosure.

FIG. 6A illustrates a depiction of a step performed during an implantation procedure in accordance with embodiments of the present disclosure.

FIG. 6B illustrates a depiction of another step performed during an implantation procedure in accordance with embodiments of the present disclosure.

FIG. 6C illustrates a depiction of another step performed during an implantation procedure in accordance with embodiments of the present disclosure.

FIG. 6D illustrates a depiction of another step performed during an implantation procedure in accordance with embodiments of the present disclosure.

FIG. 6E illustrates a depiction of another step performed during an implantation procedure in accordance with embodiments of the present disclosure.

FIG. 6F illustrates a depiction of another step performed during an implantation procedure in accordance with embodiments of the present disclosure.

FIG. 6G illustrates a depiction of another step performed during an implantation procedure in accordance with embodiments of the present disclosure.

FIG. 7 illustrates another implant in accordance with embodiments of the present disclosure.

FIG. 8 illustrates a flow diagram of a method of delivering an implant to a prostate in accordance with embodiments of the present disclosure.

The drawing figures are not necessarily to scale. Certain features and components may be shown exaggerated in scale or in schematic form and some details may not be shown in the interest of clarity and conciseness.

### DETAILED DESCRIPTION

The present devices, systems, and associated methods provide clinicians with a means to treat an enlarged prostate, which may be a symptom of benign prostatic hyperplasia or hypertrophy, to alleviate its impingement on the adjacent prostatic urethra. Implants disclosed herein can be placed using a method for compressing a prostate gland or portion thereof according to the following description.

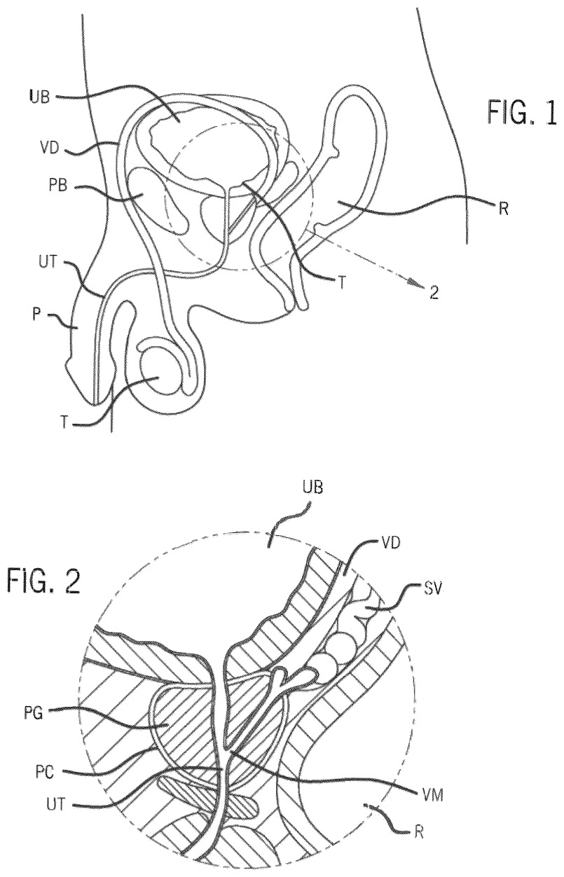

FIGS. 1 and 2 illustrate various features of the urological anatomy of a human subject. The prostate gland PG is a walnut-sized muscular gland located adjacent the urinary bladder UB. The urethra UT runs through the prostate gland PG. The prostate gland PG secretes fluid that protects and nourishes sperm. The prostate also contracts during sperm ejaculation to expel semen and provide a valve to keep urine out of the semen. A firm prostatic capsule PC surrounds the prostate gland PG.

The urinary bladder UB holds urine. The vas deferentia VD define ducts through which semen is carried, and the seminal vesicles SV secrete seminal fluid. The rectum R is the end segment of the large intestine through which waste is dispelled. The urethra UT carries both urine and semen out of the body. Thus, the urethra is connected to the urinary bladder UB and provides a passageway to the vas deferentia VD and seminal vesicles SV.

The trigone T is a smooth triangular end of the bladder. It is sensitive to expansion and signals the brain when the urinary bladder UB is full. The verumontanum VM is a crest in the wall of the urethra UT where the seminal ducts enter. The prostatic urethra is the section of the urethra UT that extends through the prostate.

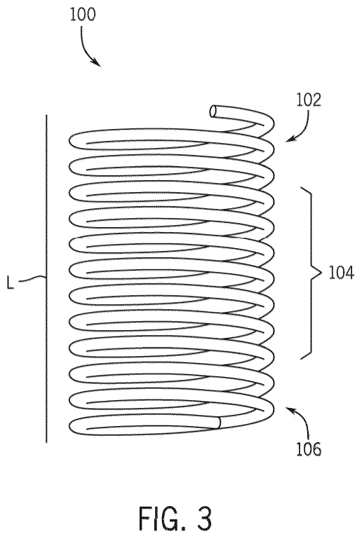

FIG. 3 illustrates an embodiment of an implant 100 used to compress at least a portion of a prostate gland in a subject. The implant 100 may be a single-component, self-tensioning coil member comprising a helical wire body resembling a spring. The implant 100 may include one or more zones or portions defined by distinct material properties, configurations, and/or anatomical placements after implantation of the device in a prostate gland. The example shown includes a distal portion 102, a middle portion 104, and a proximal portion 106. Longitudinal extension of the implant 100 creates tension, such that the distal portion 102 and proximal portion 106 are biased toward each other via the extended middle portion 104. FIG. 3 depicts the implant 100 in a collapsed, resting state.

Additional embodiments may include fewer or more than three distinct portions, which may be demarcated by discrete boundaries or gradual transitions along the length L of the implant 100. As noted, the boundaries between distinct portions may be defined by the location and/or configuration of sections of the implant 100 after implantation. According to such examples, the material composition of the implant 100 may be the same or substantially the same across one or more portions. In addition or alternatively, one or more portions may be defined by distinct material compositions, shape and/or dimensions, and/or resting-state configurations, such that certain portion(s) may comprise different materials relative to other portion(s). The resulting elasticity of the implant 100 may thus vary along its length. Tension of the implant 100 may be adjusted by different material selections, material thicknesses, dimensions, and/or size of each distinct portion of the implant 100.

The implant 100 may be comprised of various materials, non-limiting examples of which may include a flexible stainless steel, a shape memory Nitinol, one or more metals or alloys, and/or a polymer of suitable elasticity. Embodiments featuring Nitinol may leverage super elastic or shape memory alloys to enable the implant to be straightened during delivery and return to a helical shape after implantation.

The number of complete rings constituting the implant 100 may vary, each complete ring comprising a full circle of the coiled implant in its native, non-extended state. Embodiments may include three or more rings, including four, five, six, seven, eight, nine, ten, 11, 12, 13, 14, 15, 16, 17, 18, 19, 20 total rings, or more. The coil and/or wire diameters may vary in view of a variety of factors, including for instance specific anatomies and prostate sizes, along with the tension necessary to sufficiently de-obstruct the prostate after implantation.

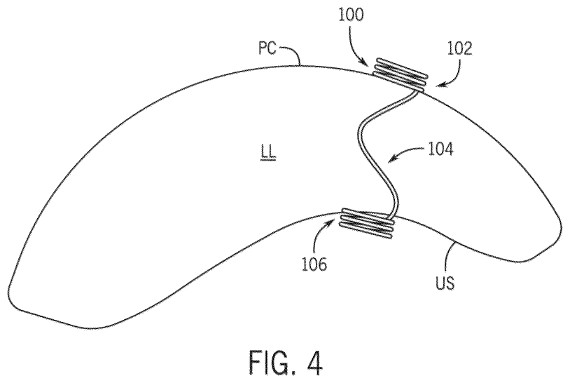

FIG. 4 is a cross-sectional view of a lateral lobe LL of a prostate gland after implantation of the implant 100. As shown, distinct zones or portions of the implant 100 may be readily discernible after implantation. The distal portion 102 of the implant 100 is anchored to the outer surface of the prostatic capsule PC of the lateral lobe LL, the proximal portion 106 is anchored to the urethral side US of the lateral lobe LL, and the middle portion 104 is extended through the lateral lobe LL. The middle portion 104 is at least partially expanded, unwound, and/or uncoiled, and the distal portion 102 and proximal portion 106 at least generally have returned to their pre-formed coil configurations configured to anchor the implant 100 to the lateral lobe LL. In the extended state shown in FIG. 4, the middle portion 104 biases the distal portion 102 and the proximal portion 106 toward each other, compressing the prostatic tissue in between the two components and alleviating constriction of the adjacent prostatic urethra.

After implantation, the distal portion 102 and proximal portion 106 may form anchors having a substantially circular footprint on the prostatic capsule PC and urethral side US of the lateral lobe, respectively. The size of each anchor footprint, represented by the “face” of the coil ring resting on the surface of the prostatic capsule PC and urethral side US of the lateral lobe LL, may be defined by the cross-sectional diameter of the implant 100 in its resting state. The implant 100 may have an advantageously large footprint relative to preexisting anchor devices, especially those lacking helical or coiled features. The implant 100 may be provided such that its cross-section is circular, generally circular, ovoid, or any other suitable geometry. The large footprint may increase the volume of prostatic tissue retracted after implantation, which may enhance the effectiveness of the implant as a whole by opening the constricted prostatic urethra to a greater extent that achieved via preexisting devices. The larger footprint may also reduce the number of implants necessary to complete a given procedure. In some examples, the large footprint may reduce the likelihood of the distal portion 102 and/or proximal portion 106 pulling through the prostatic tissue after implantation. Pull-through events may cut or tear the prostate gland and diminish its intended compression. Reduced pull-through may include a reduced pull-through frequency and/or extent relative to existing anchor devices and implants. The relative reduction in pull-through events may be observed immediately or shortly after implantation. The reduction may also be observed, and become more significant, as post-implantation time progresses. At least in part because of the increase in tissue retraction, the implants described herein may exert compression forces sufficient to compress an enlarged prostate gland to its original size, i.e., a size that does not impinge on the prostatic urethra.

Implants having different configurations and properties may be used for different cases. For instance, a self-tensioning implant of relatively high stiffness (and thus greater tension) may be used to compress an enlarged prostate gland of relatively high stiffness, whereas a less stiff, but similarly enlarged prostate gland may be compressed using an implant of lower stiffness (and thus lower tension). Additional properties of an implant may be adjusted to match the properties of an enlarged prostate gland. To avoid pull-through events, for example, higher-tensioned implants may be configured to have larger cross-sectional diameters, and thus anchor footprints, relative to implants having lower tension levels. Accordingly, methods of treating enlarged prostate glands may involve determining one or more properties of an enlarged prostate gland, such as size (including coil and wire diameters) and/or stiffness, and selecting an implant based on the properties. Other embodiments may feature a “one-size-fits-all” implant configured to accommodate any or most prostate glands.

In some cases, implant selection may also involve determining the number of implants to employ, with more implants typically required for larger, longer, and/or softer prostate glands. The large footprint of the implants disclosed herein may reduce the number of implants necessary per enlarged lobe, such that one of the disclosed implants may provide substantially the same or even greater tissue retraction and compression force relative to two or more differently configured implants, including those having smaller footprints.

After implantation, the number of complete spirals or rings constituting the distal portion 102 and the proximal portion 106 of the implant 100 may vary. In some examples, one or both portions may include at least one complete ring, including two, three, four, five rings, or more. Less than one complete ring may be sufficient to anchor the implant in other embodiments, for example such that about 50%, 60%, 70%, 80%, 90%, 95%, or more of a complete ring may suffice. The number of complete rings constituting the distal portion 102 and/or proximal portion 106 may be predefined, such that the number of rings constituting each portion may be independent of the characteristics of the targeted lobe, such as its size and stiffness.

In other embodiments, the number of complete rings constituting the distal portion 102 and/or proximal portion 106 may vary depending on the characteristics of the targeted lobe and/or implantation procedure. For example, the distal and proximal portions 102, 106 of the implant 100 may comprise fewer coils after implantation in a large lobe relative to a smaller lobe. The middle portion 104 of the implant 100 may be further extended in such examples to span the entire thickness of the lobe.

The number of complete rings constituting each anchor may be independent of the targeted lobe characteristics in embodiments featuring an implant comprised of different material compositions along the length of the implant, each unique material composition defining one or more discrete portions of the implant having, in some examples, varying flexibility levels. The number of complete rings constituting each anchor may vary in response to different characteristics of the targeted lobe in embodiments featuring an implant comprised entirely of one material composition having uniform flexibility. Additionally or alternatively, the number of coils necessary to provide a sufficient anchor may depend on the strength or stiffness of the material composition of the implant 100 such that an implant comprised in whole or in part of a more rigid material, for example, may require the formation of fewer coils relative to an implant comprising more flexible material(s). In some examples, the distal portion 102 and the proximal portion 106 may comprise a first material composition, and the middle portion 104 may comprise second material composition, different than the first material composition. According to such examples, the first material composition may comprise a metal or polymer structure, and the middle portion 104 may comprise an clastic polymer, suture, and/or generally straightened coil wire.

Embodiments of the implant 100 may also feature one or more markers used to determine whether insertion of the implant 100 is complete. For instance, the demarcation between the distal portion 102 and the middle portion 104 may feature a radiopaque marker visible only after reaching the outer surface of the prostatic capsule PC. Detection of the marker indicates that the distal portion 102 has been sufficiently advanced, thereby allowing the capsular anchor to completely form, i.e., to re-attain the pre-formed coil(s).

The implant 100 may be delivered to a targeted prostate gland using a delivery system that further includes a delivery device comprising a tubular elongate member and at least one projecting needle configured to be advanced therethrough. The needle may have a sharp distal tip configured to pierce the prostate gland, including the outer capsule.

Examples of the delivery device may generally include a handle assembly supporting an elongate portion comprising a tubular elongate member in the form of or comprising a shaft. The elongate member may be substantially rigid or flexible and defines a low profile suited to navigate body anatomy to reach an interventional site. Substructure may be provided to maintain a longitudinal profile of the elongate member so that the interventional procedure can progress as intended. Embodiments of the delivery device may also include an endoscope, providing the ability to view the interventional procedure. Using the disclosed systems, insertion of one or more implants in a prostate gland may be performed in an outpatient setting. In some examples, the delivery device may be configured to deliver multiple, discrete implants originally provided in the form of a single, long coil that is subsequently cut one or more times to form the implants. The single, long coil (relative to the length of each individual implant), may be provided in a single cartridge coupled with the handle assembly in some cases. The long coil may be cut after each separate implant is delivered, thereby allowing multiple implants to be delivered in the prostatic tissue without having to withdraw the tubular elongate member. According to such embodiments, the delivery device can continue feeding a wire, predisposed to coiling, through the device as the wire implants are formed during a procedure.

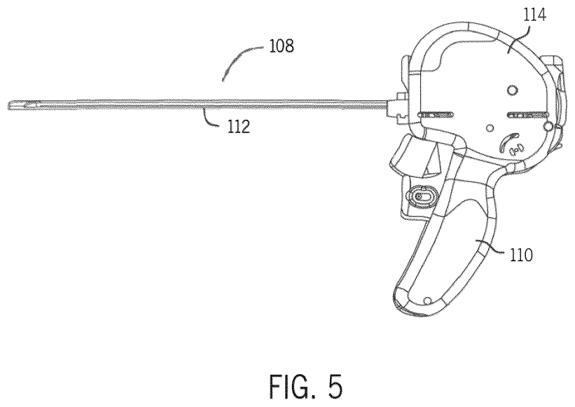

FIG. 5 illustrates one example of a delivery device 108 having structure configured to gain access to an interventional site and deploy a prostatic implant, such as implant 100. As shown, the delivery device 108 may include a handle assembly 110 connected to a tubular elongate member 112. The elongate member 112 may be sized to fit within a 19 F cystoscopic sheath for patient tolerance during a procedure in which the subject is awake rather than under general anesthesia.

The delivery device 108 may further include a number of subassemblies configured to deliver and employ an implant at a target site. A handle case assembly 114 may be included, including handle parts that form part of the handle assembly 110. The handle assembly 110 is sized and shaped to fit comfortably within an operator's hand and can be formed from conventional materials. Windows can be formed in the handle case assembly 114 to provide access to internal mechanisms of the device so that a manual override is available to the operator in the event the interventional procedure needs to be abandoned.

The elongate member 112 defines at least one inner lumen sized and configured to accommodate longitudinal insertion of at least a hollow delivery needle and prostatic implant therethrough. Non-limiting embodiments of the elongate member 112 may be sized to fit within a 19 Fr, 20 Fr, or 21 Fr sheath, or any other suitable size, including for example a 15 Fr, 16 Fr, 17 Fr, or 18 Fr sheath, or larger. The elongate member 112 may have a shape and/or flexibility configuring it to navigate through a urethra without kinking or puncturing the urethral wall. In some examples, the elongate member 112 may be substantially rigid, such that it maintains an approximately straight configuration during its insertion through the urethra. According to such examples, the distal portion of the elongate member 112 may be angled toward or away from various anatomical features surrounding the urethra, e.g., one or more lobes of the prostate gland, by adjusting the angular orientation of the proximal end of the elongate member 112 outside the body. The distal end of the elongate member 112 may comprise smooth, blunt, and/or beveled surfaces to avoid puncturing the urethral wall, especially upon adjusting its angular orientation after insertion.

Embodiments of the delivery device may include a variety of additional or substitute components and subassemblies, such as those described in U.S. Pat. No. 10,130,353, the entire contents of which are incorporated by reference herein.

Prior to insertion of any components of the delivery system, the implant recipient may undergo a regimen of antibiotics. Local anesthesia can be employed for the interventional procedure. A combination of an oral analgesic with a sedative or hypnotic component can be ingested by the subject. A topical anesthesia such as lidocaine liquids or gel can be applied to the bladder and urethra.

Examples of the implantation process may generally involve advancing the distal end of a tubular elongate member containing a coaxial needle through the urethra of a subject until the distal end reaches the prostatic urethra, adjacent one or more lobes of the enlarged prostate gland targeted for compression. Such lobes may include a lateral lobe or median lobe. A prostatic implant (e.g., implant 100) is positioned within the needle in an extended, at least partially uncoiled configuration that is generally but not necessarily fully straightened. The distal end of the needle is advanced through and beyond the distal end of the elongate member, subsequently piercing through the urethral wall, targeted prostatic lobe, and outer prostatic capsule.

The needle may then be retracted proximally toward the urethra, where the elongate member remains. As the needle is retracted, the prostatic implant is unsheathed in a distal-to-proximal direction, such that the distal portion of the implant is unsheathed first, outside the prostatic capsule. No longer bound within the inner lumen of the needle, the distal portion of the implant returns to its pre-formed, coiled configuration, anchoring the implant to the capsular side of the lobe. The middle portion of the implant is then unsheathed within the lobe, where it remains in an extended, at least partially uncoiled configuration. As the distal end of the needle further retracts back into the urethra, the proximal portion of the implant is unsheathed on the urethral side of the lobe, where it also reforms its helical shape, resulting in a wind-up effect that applies tension to the prostatic tissue sufficient to compress the lobe. With the implant fully deployed, the needle is retracted back into the elongate member, which may then be retracted proximally through the urethra until exiting the subject.

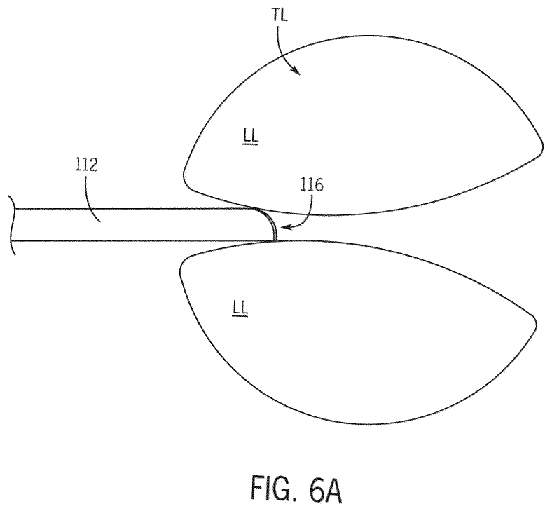

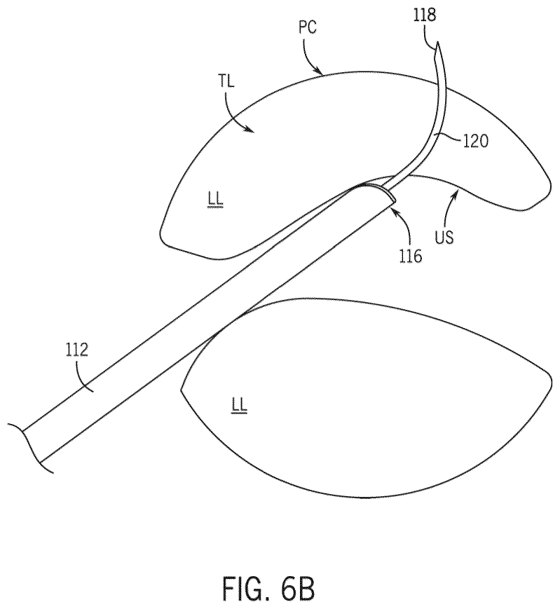

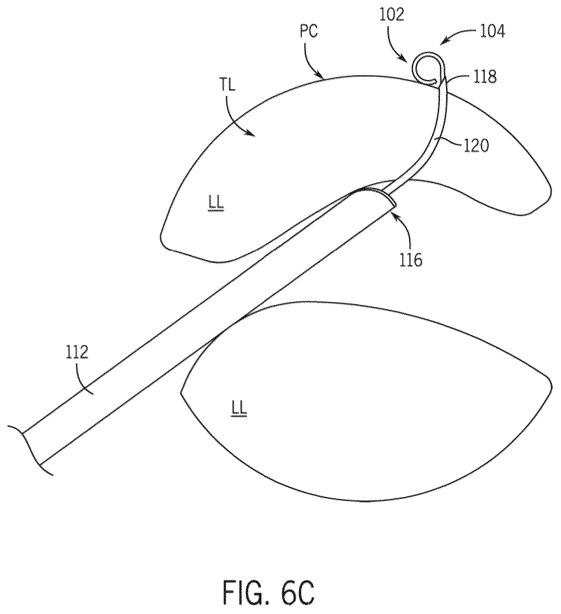

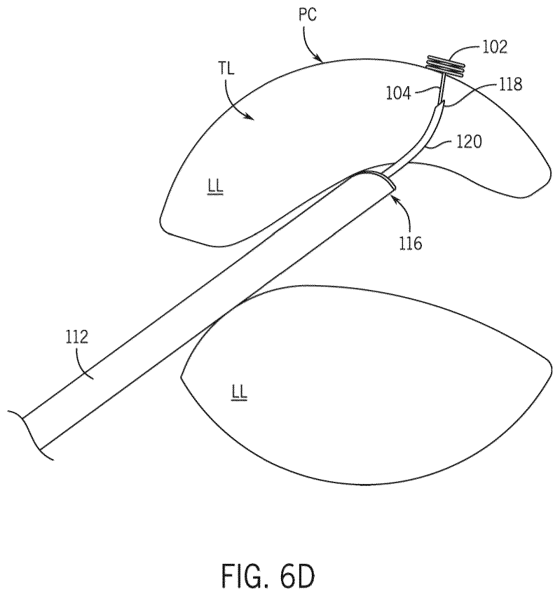

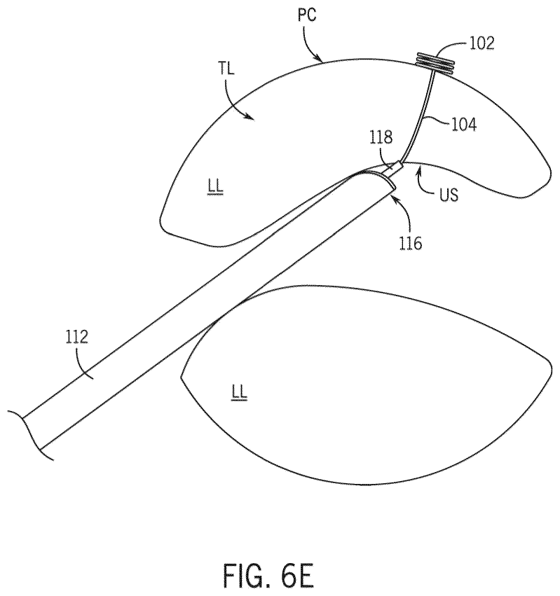

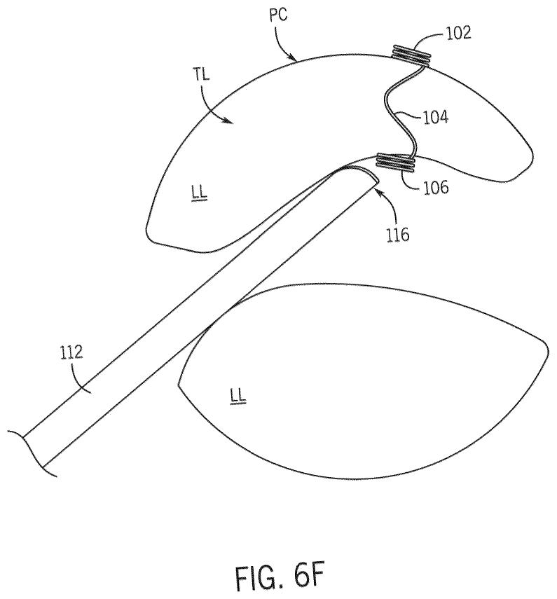

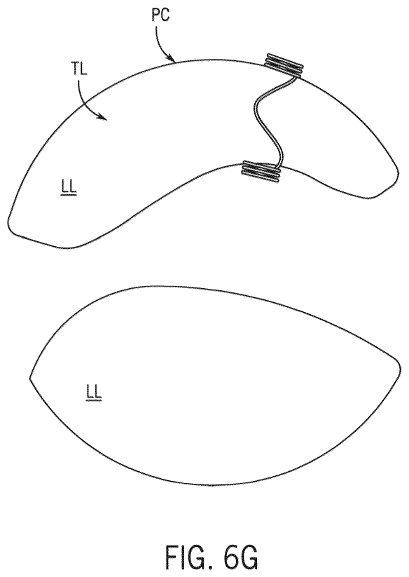

FIG. 6A illustrates a step in an example procedure for deploying a prostatic implant in a subject in accordance with embodiments disclosed herein. After preparing the treatment site for the procedure, the distal end 116 of the elongate member 112 may be inserted into the urethra (directly or within a pre-placed introducer sheath) and advanced distally therethrough, toward the urinary bladder, until it reaches the prostatic urethra, between lateral lobes LL of the prostate gland, at least one of which may be an enlarged targeted lobe TL. In some examples, the targeted lobe TL of the prostate gland is chosen while the device extends through at least a portion of the prostatic urethra. In other embodiments, the targeted lobe TL is identified prior to the procedure, for example via ultrasound imaging. A distal portion of the elongate member 112 may, in some examples, be advanced into the bladder, where it may be positioned and/or rotated as needed to deploy the implant as desired within the targeted lobe TL upon retracting the elongate member 112 until its distal end 116 returns to the prostatic urethra.

As shown in FIG. 6B, the operating clinician may use the distal end 116 of the elongate member 112 to apply lateral pressure to the targeted lobe TL from within the prostatic urethra. Adjusting the angular orientation of the distal end 116 relative to the targeted lobe TL may be achieved by adjusting the position of the proximal end of the elongate member 112. Substantially rigid embodiments of the elongate member 112 may be particularly well suited for angular adjustments of the component effected in this manner. Additional embodiments may include a compression structure, e.g., spring-loaded platform, configured to apply pressure to the targeted lobe TL without, or in addition to, adjusting the angular orientation of the elongate member.

The distal tip 118 of a delivery needle 120 may then be advanced distally through an opening defined by the distal end 116 of the elongate member 112. The clinician continues to advance the needle 120 distally until the distal tip 118 pierces the urethral side of the targeted lobe TL, passes through the prostatic tissue, and ultimately pierces the outer prostatic capsule PC.

After at least the distal tip 118 extends beyond the prostatic capsule PC, the needle 120 may be retracted proximally while the implant positioned therein is not retracted, as shown in FIG. 6C, resulting in the needle 120 unsheathing the implant 100 in a distal-to-proximal direction, starting with the distal portion 102 of the implant 100, which is unsheathed outside the prostatic capsule PC. Free from the constraints of the needle 120, the distal portion 102 begins to re-coil. The nascent coil may emerge from the needle 120 in an angular, orthogonal or approximately perpendicular orientation relative to the surface of the prostatic capsule PC, as shown (it also may emerge in any other configuration or orientation relative to the PC). In some examples, advancement of the distal portion 102 through and beyond the targeted lobe TL is not complete until at least one complete ring is formed on the prostatic capsule PC upon retraction of the needle 120. Coil formation may thus inform the implantation process such that the implant 100 is advanced distally until at least a certain number of ring(s) (including less than one full ring) are formed by the distal portion 102 upon retraction of the delivery needle 120.

FIG. 6D illustrates the distal portion 102 of the implant 100 after returning to its pre-formed coil configuration, which forms a capsular anchor configured to resist or prevent proximal movement of the implant 100. As shown, the fully formed anchor may comprise one or more complete rings arranged generally parallel to the surface of the prostatic capsule PC. The surface of the prostatic capsule PC may not be uniformly flat or planar, however, such that the coils of the distal portion 102 may not be perfectly or even substantially parallel to or flush with the prostatic capsule PC. The relative orientation between the distal portion 102 and the prostatic capsule PC may thus depend on the surface features of the capsule.

Continued retraction of the needle 120 unsheathes a portion of the middle portion 104 of the implant 100 within the targeted lobe TL. The needle 120 is further retracted until its distal tip 118 is retracted through the urethral side US, as shown in FIG. 6E. Full retraction of the needle 120 from the targeted lobe TL completes the release, e.g., unsheathing, of the proximal portion 106 of the implant 100, thus allowing the proximal portion 106 to return to its pre-formed coiled configuration and form the urethral side end-piece or anchor shown in FIG. 6F. Over-insertion of the implant 100 may be evidenced by less than one complete ring forming on the urethral side US of the targeted lobe TL.

The needle 120 may be retracted into the elongate member 112 and the two components retracted proximally through the urethra, leaving the implant 100 at the targeted lobe TL (FIG. 6G), where the extended, partially uncoiled middle portion 104 biases the distal portion 102 and proximal portion 106 toward each other, thereby compressing the prostatic tissue. The inherent tension of the implant 100 may maintain a constant, permanent compression force on the targeted lobe TL. The implant 100 may not loosen over time, either via stretching and/or remodeling of the targeted lobe into a more relaxed, compressed state, thus enhancing the long-term therapeutic effect of the implant 100. As the targeted lobe TL contracts or is reduced over time, the implant 100 maintains its tension on the lobe, thus maintaining and potentially improving the therapeutic effect of the implant 100.

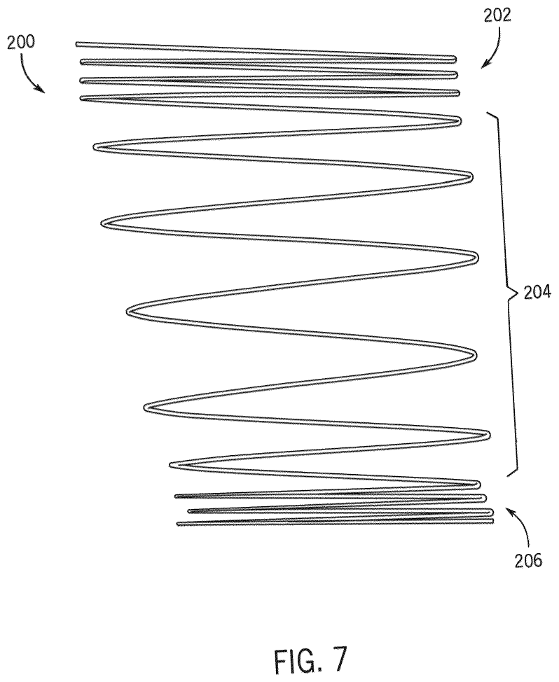

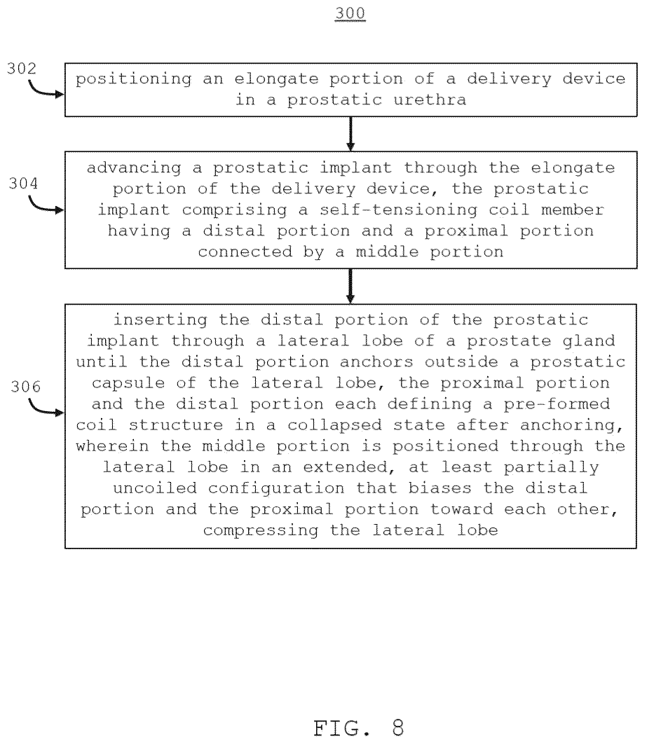

As noted herein, the characteristics of the prostatic implant may vary along its length. As shown in FIG. 7, for instance, different segments of an implant 200 may be provided with different levels of coil tightness. The implant 200 may include a distal portion 202, a middle portion 204, and a proximal portion 206, where the distal and proximal portions are tighter than the middle portion in the relaxed, native state of the device. This particular embodiment may provide less tension than a coiled implant having substantially uniform tightness. The implant 200 may exert a constant tension between the distal portion 202 and the proximal portion 206, but the amount of tension may be controlled or reduced. The three zones also can vary in other ways, e.g., the middle portion 204 can be made of a more flexible material, be of a different diameter, have a different elasticity, or combinations of these as compared to distal portion 202 and/or proximal portion 206. In some examples, the middle portion 204 may be straight or substantially straight, while the distal portion 202 and proximal portion 206 are predisposed to coiling.

FIG. 8 is a flow diagram of a non-limiting example of a method 300 of delivering an implant to a prostate gland in accordance with embodiments of the present disclosure. The example method 300 may be performed in accordance with embodiments of the present disclosure. The example method 300 may be performed to treat or alleviate symptoms of an enlarged prostate gland, which may be caused by a variety of conditions, including but not limited to benign prostatic hyperplasia and/or benign prostatic hypertrophy, along with cancerous conditions that increase the size of the prostate gland, for example via tumor formation. The method may be implemented by one or more of the disclosed devices, systems, and/or components thereof. One or more additional steps may be included, and certain steps may also be omitted or performed in a different sequence than depicted.

In the embodiment shown, the method 300 begins at step 302 by “positioning an elongate portion of a delivery device in a prostatic urethra. At step 304, the method involves “advancing a prostatic implant through the elongate portion of the delivery device, the prostatic implant comprising a self-tensioning coil member having a distal portion and a proximal portion connected by a middle portion.” At step 306, the method involves “inserting the distal portion of the prostatic implant through a lateral lobe of a prostate gland until the distal portion anchors outside a prostatic capsule of the lateral lobe, the proximal portion and the distal portion each defining a pre-formed coil structure in a collapsed state after anchoring, wherein the middle portion is positioned through the lateral lobe in an extended, at least partially uncoiled configuration that biases the distal portion and the proximal portion toward each other, compressing the lateral lobe.”

### EXAMPLES

The above Detailed Description includes references to the accompanying drawings, which form a part of the Detailed Description. The Detailed Description should be read with reference to the drawings. The drawings show, by way of illustration, specific embodiments in which the present accessory devices and associated methods can be practiced. These embodiments are also referred to herein as “examples.”

The Detailed Description is intended to be illustrative and not restrictive. For example, the above-described examples (or one or more features or components thereof) can be used in combination with each other. Other embodiments can be used, such as by one of ordinary skill in the art upon reviewing the above Detailed Description. Also, various features or components have been or can be grouped together to streamline the disclosure. This should not be interpreted as intending that an unclaimed disclosed feature is essential to any claim. Rather, inventive subject matter can lie in less than all features of a particular disclosed embodiment. Thus, the following claim examples are hereby incorporated into the Detailed Description, with each example standing on its own as a separate embodiment:

In Example 1, a method for treating benign prostatic hyperplasia (or an otherwise enlarged prostate) involves positioning an elongate portion of a delivery device in a prostatic urethra; advancing a prostatic implant through the elongate portion of the delivery device, the prostatic implant comprising a self-tensioning coil member having a distal portion and a proximal portion connected by a middle portion; and inserting the distal portion of the prostatic implant through a lateral lobe of a prostate gland until the distal portion anchors outside a prostatic capsule of the lateral lobe, the proximal portion and the distal portion each defining a pre-formed coil structure in a collapsed state after anchoring, wherein the middle portion is positioned through the lateral lobe in an extended, at least partially uncoiled configuration that biases the distal portion and the proximal portion toward each other, compressing the lateral lobe.

In Example 2, the implant may be a single component of unitary construction.

In Example 3, each pre-formed coil structure may define a face and a circular edge, the face resting on the prostatic capsule and defining an anchoring footprint of the prostatic implant.

In Example 4, the prostatic implant may comprise a uniform material composition.

In Example 5, the prostatic implant may comprise two or more distinct material compositions.

In Example 6, the distal portion and the proximal portion may comprise a first material composition, and the middle portion may define a second material composition, different than the first.

In Example 7, the prostatic implant may comprise one or more metals.

In Example 8, the prostatic implant may comprise one or more elastic polymers.

In Example 9, inserting the distal portion of the prostatic implant through the lateral lobe of the prostate gland may comprise inserting a hollow delivery needle containing the prostatic implant through the lateral lobe.

In Example 10, the method may further involve unsheathing the prostatic implant by retracting the hollow needle in a proximal direction.

In Example 11, the prostatic implant may exit the elongate portion of the delivery device through an opening defined by a distal tip of the elongate portion.

In Example 12, the method further involves determining a number of rings constituting the pre-formed coil structure outside the prostatic capsule.

In Example 13, sufficient insertion of the prostatic implant may require a presence of a pre-defined number of rings in the pre-formed coil structure outside the prostatic capsule.

In Example 14, the prostatic implant may comprise a uniform level of coil tightness.

In Example 15, the prostatic implant may comprise two or more levels of coil tightness.

In Example 16, the method may further involve using a distal end of the elongate portion to apply a pressure to the lateral lobe from within the prostatic urethra.

In Example 17, a system for treating benign prostatic hyperplasia includes a delivery device comprising a handle assembly connected to an elongate portion, the elongate portion comprising a tubular member defining an inner lumen. The system may also include a hollow delivery needle having a sharp distal tip and configured to be extended through the inner lumen of the tubular member upon activation of the handle assembly. The system may also include a prostatic implant comprising a self-tensioning coil member having a distal portion and a proximal portion connected by a middle portion, the prostatic implant configured to be extended through the hollow delivery needle in a partially uncoiled configuration. The distal portion of the prostatic implant may be configured to be inserted through a lateral lobe of a prostate gland, led by the sharp distal tip of the hollow delivery needle, until the distal portion anchors outside a prostatic capsule of the lateral lobe upon retraction of the hollow delivery needle in a proximal direction into the lateral lobe. The proximal portion and the distal portion may each define a pre-formed coil structure in a collapsed state after anchoring.

In Example 18, the middle portion may be positioned through the lateral lobe in an extended, at least partially uncoiled configuration that biases the distal portion and the proximal portion toward each other, compressing the lateral lobe, upon further retraction of the hollow delivery needle proximal to a urethral side of the lateral lobe.

In Example 19, the elongate portion may be substantially rigid, such that a change in an angular orientation of a proximal end of the elongate portion causes a corresponding change in an angular orientation of a distal end of the elongate portion.

In Example 20, the implant may be a single component of unitary construction.

In Example 21, a prostatic implant configured to compress an enlarged prostate includes a self-tensioning coil member having a distal portion and a proximal portion connected by a middle portion, the prostatic implant configured to be extended through the hollow delivery needle in a partially uncoiled configuration.

In Example 22, the prostatic implant of Example 21 may be configured such that the distal portion of the prostatic implant is configured to be inserted through a lateral lobe of a prostate gland, led by the sharp distal tip of the hollow delivery needle within which the prostatic implant may be partially or fully enclosed during its delivery.

In Example 23, the prostatic implant of Example 22 may be configured such that the proximal portion and the distal portion each define a pre-formed coil structure in a collapsed state after anchoring.

In Example 24, a prostatic implant comprises a self-tensioning coil member, which may have a distal portion and a prostatic portion connected by a middle portion. The prostatic implant may be configured to be positioned within and extended through a hollow needle in a partially uncoiled configuration. The distal portion of the prostatic implant may be configured to be inserted through a lobe of a prostate gland, led by the sharp distal tip of the delivery needle. The proximal portion and distal portion of the prostatic implant may each define a pre-formed coil structure in a collapsed state when at rest, e.g., when unconfined by a hollow needle or other structure, which may have a cross-sectional width that is less than the cross-sectional dimension of the implant in its natural state.

### CLOSING NOTES

Certain terms are used throughout this patent document to refer to particular features or components. As one skilled in the art appreciates, different people may refer to the same feature or component by different names. This patent document does not intend to distinguish between components or features that differ in name but not in function.

For the following defined terms, certain definitions shall be applied unless a different definition is given elsewhere in this patent document. The terms “a,” “an,” and “the” are used to include one or more than one, independent of any other instances or usages of “at least one” or “one or more.” The term “or” is used to refer to a nonexclusive or, such that “A or B” includes “A but not B,” “B but not A,” and “A and B.” All numeric values are assumed to be modified by the term “about,” whether or not explicitly indicated. The term “about” generally refers to a range of numbers that one of skill in the art would consider functionally equivalent to the recited value (e.g., having the same function or result). In many instances, the term “about” can include numbers that are rounded to the nearest significant figure. The recitation of numerical ranges by endpoints includes all numbers and sub-ranges within and bounding that range (e.g., 1 to 4 includes 1, 1.5, 1.75, 2, 2.3, 2.6, 2.9, etc. and 1 to 1.5, 1 to 2, 1 to 3, 2 to 3.5, 2 to 4, 3 to 4, etc.). The terms “patient” and “subject” are intended to include mammals, such as for human or veterinary applications. The terms “distal” and “proximal” are used to refer to a position or direction relative to the treating clinician. “Distal” and “distally” refer to a position that is distant from, or in a direction away from, the treating clinician. “Proximal” and “proximally” refer to a position that is near, or in a direction toward, the treating clinician.

The scope of the invention should be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended; that is, a device, kit or method that includes features or components in addition to those listed after such a term in a claim are still deemed to fall within the scope of that claim. Moreover, in the following claims, the terms “first,” “second” and “third,” etc. are used merely as labels, and are not intended to impose numerical requirements on their objects.

The Abstract is provided to allow the reader to quickly ascertain the nature of the technical disclosure. It is submitted with the understanding that it will not be used to interpret or limit the scope or meaning of the claims.

What is claimed is:

1. A method for treating benign prostatic hyperplasia, comprising:
positioning an elongate portion of a delivery device in a prostatic urethra;
advancing a prostatic implant through the elongate portion of the delivery device, the prostatic implant comprising a self-tensioning coil member having a distal portion and a proximal portion connected by a middle portion; and
inserting the distal portion of the prostatic implant through a lateral lobe of a prostate gland until the distal portion anchors outside a prostatic capsule of the lateral lobe, the proximal portion and the distal portion each defining a pre-formed coil structure in a collapsed state after anchoring, wherein the middle portion is positioned through the lateral lobe in an extended, at least partially uncoiled configuration that biases the distal portion and the proximal portion toward each other, compressing the lateral lobe.

2. The method of claim 1, wherein the implant is a single component of unitary construction.

3. The method of claim 1, wherein each pre-formed coil structure defines a face and a circular edge, the face resting on the prostatic capsule and defining an anchoring footprint of the prostatic implant.

4. The method of claim 1, wherein the prostatic implant comprises a uniform material composition.

5. The method of claim 1, wherein the prostatic implant comprises two or more distinct material compositions.

6. The method of claim 5, wherein the distal portion and the proximal portion comprise a first material composition, and the middle portion defines a second material composition, different than the first.

7. The method of claim 1, wherein the prostatic implant comprises one or more metals.

8. The method of claim 1, wherein the prostatic implant comprises one or more elastic polymers.

9. The method of claim 1, wherein inserting the distal portion of the prostatic implant through the lateral lobe of the prostate gland comprises inserting a hollow delivery needle containing the prostatic implant through the lateral lobe.

10. The method of claim 9, further comprising unsheathing the prostatic implant by retracting the hollow needle in a proximal direction.

11. The method of claim 1, wherein the prostatic implant exits the elongate portion of the delivery device through an opening defined by a distal tip of the elongate portion.

12. The method of claim 1, further comprising determining a number of rings constituting the pre-formed coil structure outside the prostatic capsule.

13. The method of claim 12, wherein sufficient insertion of the prostatic implant requires a presence of a pre-defined number of rings in the pre-formed coil structure outside the prostatic capsule.

14. The method of claim 1, wherein the prostatic implant comprises a uniform level of coil tightness.

15. The method of claim 1, wherein the prostatic implant comprises two or more levels of coil tightness.

16. The method of claim 1, further comprising using a distal end of the elongate portion to apply a pressure to the lateral lobe from within the prostatic urethra.

17. A system for treating benign prostatic hyperplasia, comprising:
a delivery device comprising a handle assembly connected to an elongate portion, the elongate portion comprising a tubular member defining an inner lumen;
a hollow delivery needle having a sharp distal tip and configured to be extended through the inner lumen of the tubular member upon activation of the handle assembly; and
a prostatic implant comprising a self-tensioning coil member having a distal portion and a proximal portion connected by a middle portion, the prostatic implant configured to be extended through the hollow delivery needle in a partially uncoiled configuration,
wherein the distal portion of the prostatic implant is configured to be inserted through a lateral lobe of a prostate gland, led by the sharp distal tip of the hollow delivery needle, until the distal portion anchors outside a prostatic capsule of the lateral lobe upon retraction of the hollow delivery needle in a proximal direction into the lateral lobe, and
wherein the proximal portion and the distal portion each define a pre-formed coil structure in a collapsed state after anchoring.

18. The system of claim 17, wherein the middle portion is positioned through the lateral lobe in an extended, at least partially uncoiled configuration that biases the distal portion and the proximal portion toward each other, compressing the lateral lobe, upon further retraction of the hollow delivery needle proximal to a urethral side of the lateral lobe.

19. The system of claim 17, wherein the elongate portion is substantially rigid, such that a change in an angular orientation of a proximal end of the elongate portion causes a corresponding change in an angular orientation of a distal end of the elongate portion.

20. The system of claim 17, wherein the implant is a single component of unitary construction.
